(19) United States
(12) Patent Application Publication
(10) Pub. No.: US 2025/0339054 A1
(43) Pub. Date: Nov. 6, 2025

(54) SYSTEMS AND METHODS FOR TREMOR DETECTION IN HEARING DEVICES

(71) Applicant: Starkey Laboratories, Inc., Eden Prairie, MN (US)

(72) Inventors: Delian Penev Delev, Eden Prairie, MN (US); Andy Shih-Hsuan Lin, Chanhassen, MN (US); Kyle Olson, St. Louis Park, MN (US)

(21) Appl. No.: 19/192,667

(22) Filed: Apr. 29, 2025

Related U.S. Application Data
(60) Provisional application No. 63/641,677, filed on May 2, 2024.

(51) Int. Cl. A61B 5/11; A61B 5/00

(52) U.S. Cl. CPC A61B 5/1101; A61B 5/6803; A61B 5/6844; A61B 5/746; A61B 2560/0214; A61B 2562/0219

(57) ABSTRACT

Disclosed herein, among other things, are systems and methods for tremor detection in hearing devices. A method includes receiving a signal from a motion sensor, such as an inertial measurement unit (IMU), of the hearing device. Upon determining the hearing device has been removed from an ear of a user, recording the signal from the motion sensor begins. Upon determining the hearing device has been placed on or in a charging device or on a surface, recording the signal from the motion sensor in the memory is discontinued. The recorded signal is used to determine whether the user has a tremor. Upon determining the user has the tremor, a notification is provided indicative of the presence of the tremor to one or more of the user or a clinician.

### RELATED APPLICATIONS

This application claims the benefit of priority to U.S. Provisional Application Ser. No. 63/641,677, filed May 2, 2024, the content of which is incorporated herein by reference in its entirety.

### TECHNICAL FIELD

This document relates generally to audio device systems and more particularly to systems and methods for tremor detection in hearing devices.

### BACKGROUND

Audio devices can be used to provide audible output to a user based on received wireless signals. Examples of audio devices include speakers and ear-wearable devices, also referred to herein as hearing devices. Example of hearing devices include hearing assistance devices or hearing instruments, including both prescriptive devices and non-prescriptive devices. Specific examples of hearing devices include, but are not limited to, hearing aids, headphones, and earbuds.

Hearing aids are used to assist patients suffering hearing loss by transmitting amplified sounds to ear canals. In one example, a hearing aid is worn in and/or around a patient's ear. Hearing aids may include processors and electronics that improve the listening experience for a specific wearer or in a specific acoustic environment.

Studies have shown that essential tremor (ET) is associated with both increased odds of prevalent dementia and increased risk of incident dementia. Currently, a patient's tremor can only be assessed by trained clinicians mostly in an infrequent in-person visit. There is no available repeatable and reliable method to monitor the condition and progress of tremor. Such a method would assist in detection of early symptoms of dementia or Alzheimer's, disorders most commonly found in elderly patients who may use hearing aids.

Thus, there is a need in the art for improved systems and methods for tremor detection in ear-wearable devices.

### SUMMARY

Disclosed herein, among other things, are systems and methods for tremor detection in hearing devices. Various aspects include a method for tremor detection in hearing devices. The method includes receiving a signal from an inertial measurement unit (IMU) sensor of the hearing device. Upon determining the hearing device has been removed from an ear of a user, recording the signal from the IMU sensor begins. Upon determining the hearing device has been placed on or in a charging device or on a surface, recording the signal from the IMU sensor in the memory is discontinued. The recorded signal is used to determine whether the user has a tremor. Upon determining the user has the tremor, a notification is provided indicative of the presence of the tremor to one or more of the user or a clinician.

Various aspects include a hearing device for tremor detection. The hearing device includes a housing, an IMU sensor on or in the housing, and hearing electronics within the housing. The hearing electronics include at least one processor and a memory including instructions that, when executed by the at least one processor, cause the at least one processor to perform operations to: receive a signal from the IMU sensor, begin recording the signal from the IMU sensor upon determining the hearing device has been removed from an ear of a user, discontinue recording the signal from the IMU sensor in the memory upon determining the hearing device has been placed on or in a charging device or on a surface, use the recorded signal to determine whether the user has a tremor, and provide a notification indicative of the presence of the tremor to one or more of the user or a clinician upon determining the user has the tremor.

This Summary is an overview of some of the teachings of the present application and not intended to be an exclusive or exhaustive treatment of the present subject matter. Further details about the present subject matter are found in the detailed description and appended claims.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates a flow diagram of method for tremor detection in hearing aids, according to various examples of the present subject matter.

FIG. 2 illustrates a flow diagram of a method for tremor detection in hearing devices, according to various examples of the present subject matter.

FIG. 3 illustrates a block diagram of a hearing device circuit, according to various examples of the present subject matter.

FIG. 4 illustrates a block diagram of an example machine upon which any one or more of the techniques discussed herein may perform.

### DETAILED DESCRIPTION

The following detailed description of the present subject matter refers to subject matter in the accompanying drawings which show, by way of illustration, specific aspects and embodiments in which the present subject matter may be practiced. These embodiments are described in sufficient detail to enable those skilled in the art to practice the present subject matter. References to “an”, “one”, or “various” embodiments in this disclosure are not necessarily to the same embodiment, and such references contemplate more than one embodiment, including combinations of such embodiments. The following detailed description is demonstrative and not to be taken in a limiting sense. The scope of the present subject matter is defined by the appended claims, along with the full scope of legal equivalents to which such claims are entitled.

The present detailed description will discuss audio devices such as hearing devices and speakers. The description refers to hearing devices or hearing instruments generally, which include earbuds, headsets, headphones, and hearing assistance devices using the example of hearing aids. Other hearing devices include, but are not limited to, those in this document. It is understood that their use in the description is intended to demonstrate the present subject matter, but not in a limited or exclusive or exhaustive sense.

Studies have shown that essential tremor (ET) is associated with both increased odds of prevalent dementia and increased risk of incident dementia. Currently, a patient's tremor can only be assessed by trained clinicians mostly in an infrequent in-person visit. There is no available repeatable and reliable method to monitor the condition and progress of tremor. Such a method would assist in detection of early symptoms of neurodegenerative diseases such as dementia or Alzheimer's, disorders most commonly found in elderly patients who may use hearing aids.

The present subject matter provides systems and methods for tremor detection for users of hearing devices. In one example, the present method detects hand tremors using a sensor (such as an IMU/accelerometer or other motion sensor) in a hearing device, such as a hearing aid or any other ear-worn device. The present subject matter uses the consistent repeatable fine motor movement/motion of a user taking the hearing device off the ear and putting the device into a charger, a movement that the user would need to do with a similar motion at a similar time every day. This repeatable motion provides a consistent base in the measurement process to provide more reliable progress monitoring and trending data. Also, because this motion is a fine motor movement which involves higher dexterity due to the small size of the ear-worn device, it is extremely useful in diagnosis of a neurodegenerative disorder using the present subject matter.

In various examples, the present subject matter may use hardware already existing on a hearing device, and does not require any new specific movement of the user, as the method becomes active when the user takes off the hearing devices at the end of every day. Thus, the present method is easy to implement and offers consistent daily data for detection of tremors and therefore prediction of dementia. The present subject matter may be implemented in a hearing aid or an earbud or any ear-worn device, in various examples.

The movement of a user taking the hearing device off the ear, and aiming for and inserting it into the charger is a consistent and repeatable movement. This movement begins with the removal of the device, holding it through the air, and ends with the insertion of the hearing aid into the charger receptacle, in one example. The motion will have a clean definition of the beginning and the end, with assessment of tremor occurring in between. Thus, the present subject matter provides a consistent evaluation baseline for evaluation and determination of the onset of tremors.

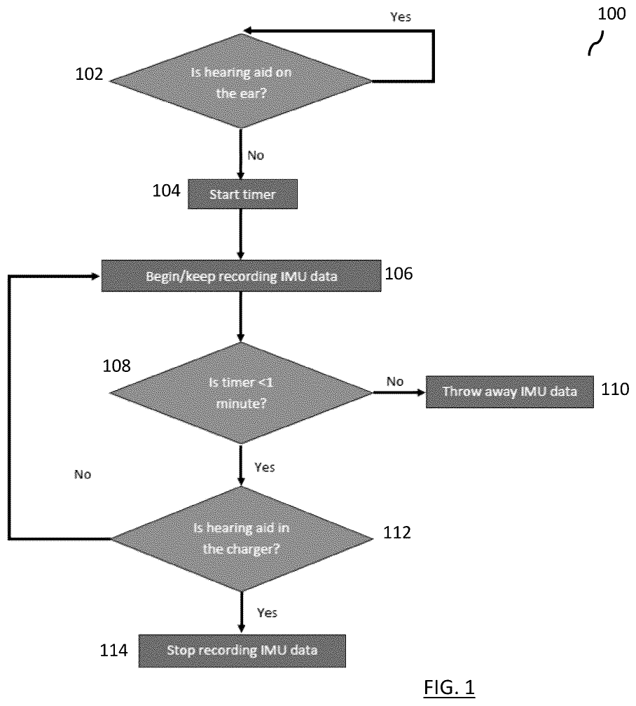

FIG. 1 illustrates a flow diagram of method for tremor detection in hearing aids, according to various examples of the present subject matter. The method 100 includes determining if the hearing aid is on or in an ear of a user, at step 102. When the hearing aid is determined to be still on or in the ear of the user, the method 100 returns to step 102 to continue with the determination. When the hearing aid is determined to be no longer on or in the ear of the user, the method 100 continues at step 104 and a timer is started. At step 106, the method 100 begins a recording of IMU data received by a processor from an IMU sensor (or other appropriate sensor, such as an accelerometer or the like) of the hearing aid. At step 108, the method 100 determines whether the timer is less than a predetermined duration, such as one minute. When the timer has exceeded the predetermined duration, the recorded IMU data is discarded at step 110. When the timer has not exceeded the predetermined duration, the method 100 determines whether the hearing aid is on or in the charger, at step 112. The method 100 may also determine whether the hearing aid is placed on a surface such as a table, or other location, in various examples. When the hearing aid is not determined to be on or in the charger, the method 100 returns to step 106 to continue recording the received sensor data. When the hearing aid is determined to be on or in the charger, the method 100 continues to step 114 to stop recording the received sensor data. In various examples, the recorded sensor data is then used to determine the presence of a tremor of the user. According to various examples, the present subject matter may evaluate the user movement in reverse, or from charger to hand to ear with the hearing device when the user puts the device on at the beginning of a day.

In various examples, the IMU sensor is used to detect removal of the device from the user's ear. In one example, an inward facing microphone is present in the device, and removal of the device from the user's ear can be detected by a change in acoustic environment as the device leaves the car. In another example, an optical proximity sensor is present in the device, as is commonly found in consumer earbuds, and the on-skin detection of the optical proximity sensor may be used to detect the removal of the device from the user's ear. In various examples, an outward microphone is included in the device, and the occlusion or covering up of the outward microphone or case noise or feedback may be used as indication of the device being touched and held by the user, hence indicating the device has been removed. Other sensors may be used without departing from the scope of the present subject matter. In some examples, a combination of sensors may be used to detect removal to make the detection process more robust.

According to various examples, insertion of the hearing device into the charger may be determined by the device's charging contacts touching the charger's contacts and establishing the connection, and identified using a power management circuit of the hearing device. Other sensors or circuits may be used to detect placement of the device on the charger or on a flat surface without departing from the scope of the present subject matter. In still other examples, the present system and method may be used with non-rechargeable hearing devices. For example, motion sensor (such as IMU sensor) data may be recorded starting from the removal of the non-rechargeable device from the user's head/ear and ending at removal of the battery from the device. In this example, upon the next power up of the non-rechargeable device (e.g., upon reinsertion of the battery), the recorded data may be analyzed to determine if a tremor has occurred.

The hearing device begins logging IMU data once the status of removal is detected, in various examples. In some examples, the logged IMU data will only be analyzed for tremor detection if the charger insertion is confirmed within a predetermined time period, such as 30 seconds or 1 minute, to remove the non-consistent data. For example, the removal status may be triggered when the user is just adjusting the device, or the device fell off the car, or the device was removed but put on the desk or in the pocket. In various examples, by clearly defining and controlling the user movement to this specific ear-to-hand-to-charger action, the tremor detection assessment is more accurate and reliable.

In various examples, once the ear-to-hand-to-charger action is confirmed and the IMU (or other sensor data) has been recorded or logged, the present subject matter performs an analysis or calculation using the logged data to detect the presence or onset of hand tremors. For example, the logged data can be assessed based on the frequency, amplitude, duration, or timing of the movements of the user. Other algorithms for tremor detection may be used without departing from the scope of the present subject matter. In addition, various algorithms may be used to predict dementia or other neurodegenerative conditions using the logged sensor data by the present system.

In another example, upon sensing being put in or on the charger, the hearing device saves IMU data from the previous 10 seconds (or other predetermined time period) to capture the latter half of the movement from ear to charger, and thus provide tremor data for the portion of the movement in which the hearing device is aimed into the receptacle. However, this portion of the movement may not always be made by the user (for example, if a user leaves their hearing aid on a table, and then later someone else puts it in the charger).

In yet another example, no sensor data is used and the tremor detection may be purely time-based, by comparing time taken by the user to move the device from ear to charger daily over time. In this example, the system records the time between taking the hearing device out of the ear and putting the hearing device in the charger, and if this time period gets longer over time, this is evidence of a tremor in between or slowed motor movements, which may be indicators of neurodegeneration. In various examples, the present subject matter may provide an audio prompt to the user using a hearing device in the other ear of the user (that has yet to be removed) that informs the user that the data is being logged. For example, the prompt may indicate that “hand tremor measurement starting” to alert the user to the recording and focus them on the task of charger insertion. In various examples, the hearing device may communicate with another device, such as a smart phone or other wireless device, to log the data, process the data, and/or provide notifications to the user. In some examples, the present method may be used in reverse, or by tracking the device from charger to hand to car.

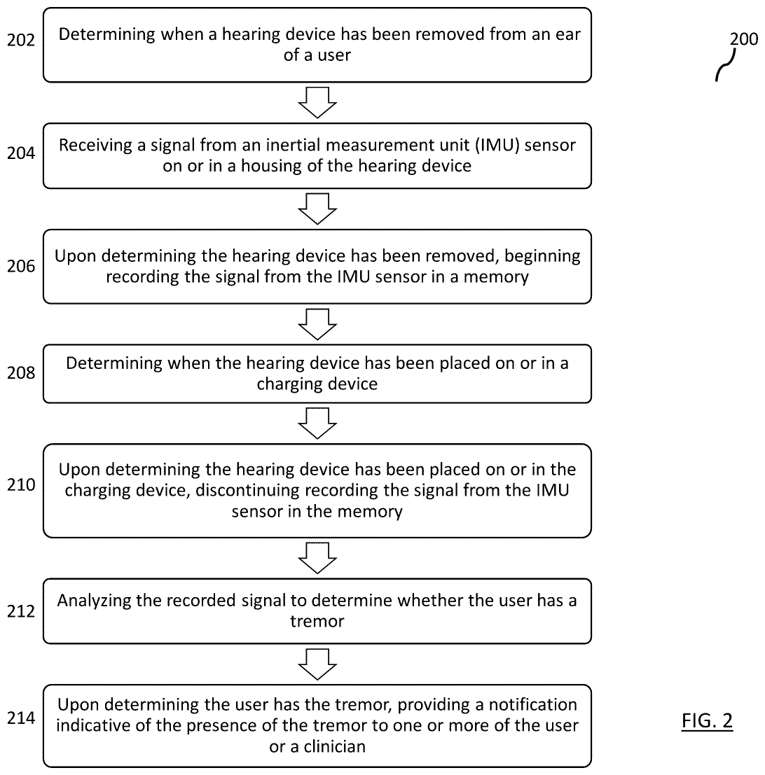

FIG. 2 illustrates a flow diagram of a method for tremor detection in hearing devices, according to various examples of the present subject matter. The method 200 includes determining when a hearing device has been removed from an car of a user, at step 202. At step 204, the method 200 includes receiving a signal from an inertial measurement unit (IMU) sensor on or in a housing of the hearing device. At step 206, upon determining the hearing device has been removed, the method 200 includes beginning recording the signal from the IMU sensor in a memory. The method 200 also includes determining when the hearing device has been placed on or in a charging device, at step 208. At step 210, upon determining the hearing device has been placed on or in the charging device, the method 200 includes discontinuing recording the signal from the IMU sensor in the memory. The method 200 further includes analyzing the recorded signal to determine whether the user has a tremor, at step 212. At step 214, upon determining the user has the tremor, the method 200 includes providing a notification indicative of the presence of the tremor to one or more of the user or a clinician.

According to various examples, determining when the hearing device has been removed from the car includes detecting a change in acoustic environment of a microphone of the hearing device. The change in acoustic environment includes occlusion or covering up of the microphone, in one example. In another example, the change in acoustic environment includes feedback from the microphone. In some examples, determining when the hearing device has been removed from the car includes using the signal from the IMU sensor. Determining when the hearing device has been removed from the car includes using a received signal from a proximity sensor, such as an optical sensor, in some examples. Other types of proximity sensors may be used without departing from the scope of the present subject matter. In various examples, determining when the hearing device has been placed on or in the charging device includes detecting when charging contacts of the hearing device touch contacts of the charging device.

According to various examples, the method may further include starting a timer upon determining the hearing device has been removed, ending the timer upon determining the hearing device has been placed on or in the charging device, comparing the timer to a predetermined time limit, and upon determining that the timer exceeds the predetermined time limit, discarding the stored signal from the IMU sensor. Various types of hearing devices may be used with the present tremor detection system, including but not limited to hearing aids.

The present subject matter provides a plurality of benefits and improvements for medical diagnostics. For example, the present system identifies symptoms of dementia in a population of hearing device users that is typically of advanced age. The present system uses motion sensors, such as IMU sensors or accelerometers that are often already present in hearing devices, thus reducing or eliminating additional costs of implementation. In addition, the present system monitors a movement by the user (removing or inserting the hearing device) that is repeated every day, and that includes a fine motor movement involving dexterity, coordination and little variation that provides a baseline for comparison to previously stored data for the user, and across other users of the same or similar devices. In some examples, the progression of symptoms or delay in movement may be recorded, and the progress tracked and used in the medical evaluation of the user. The present system ensures that the sensors used for data collection are not deactivated during the removal or insertion process, in various examples.

According to various examples, the present system processes collected data using a processor in or on the hearing device. Other processors may be used, such as in remote devices (e.g., in a smartphone) that are in communication with the hearing device. The logged data may be stored locally on the hearing device or remotely, such as on cloud storage, or using an application on mobile device. In some examples, the present system uses machine learning, including but not limited to a deep neural network or artificial intelligence, to process the tracked data. The machine learning processor, either local or remote, may be trained on data from the user and/or data from multiple users or participants, to identify tremors and/or determine how tremor symptoms (frequency, etc.) may be indicative of neurological disorders for individuals and for the general population.

The present method provides notifications of tremor identification or time delay increase, in various examples. In some examples, the present method provides notification to the user via the hearing device or in a smartphone application associated with the hearing device. The present method may also or alternatively provide notification to a healthcare provider of the user, such that the user's doctor or other individual (such as another health care provider, family member, or the like) may also keep track of patient data, symptoms, and/or tremor identification or determination.

In various examples, the present system automatically records data from one or more sensors during a time period from the user removing the hearing device from an ear until the user places the hearing device into its charger or on a surface, thus providing an auto-on and auto-off recording. The system may detect power from the charger for the auto-off recording, in an example. In some examples, an infrared proximately sensor may be used for some aspects, such as for detecting removal of the hearing device from the ear or from which ear the device was removed. Other sensors may be used without departing from the scope of the present subject matter. In various examples, an IMU sensor, an inward facing microphone, a temperature sensor temp, an ultrasound sensor, a red light sensor, a greenlight sensor, far-, near-, or mid-infrared sensors, or a capacitive sensor may be used for the auto on/off recording features. In other examples, a timer is used for the auto off feature to stop recording of motion sensor data. In one example, the motion sensor data is recorded at all times when the device is not on a charger.

In some examples, the present method may be used in reverse, or by initiating recording when the user takes the hearing device out of the charger until insertion in an ear of the user. However, the movement by the user from removal of the device from the ear to placement in charger may be more sensitive for tremor detection, as that movement is a more dexterous motion, and it is more difficult for a user to put the device in a charger than in their ear. In addition, the hearing device takes time to boot up and decrypt firmware, so if the device is coming out of the charger, there might be a boot up or delay time and recording of sensor data may not begin immediately.

In one example, to detect the presence of tremor or dementia, the present method may track how long it takes for the user to put the device into a charger by comparing the duration (logging the time from ear to charger over time) with previous durations. For example, if the user takes progressively more time to move the device from ear to charger, it may correlate to an increase in probability of tremor or dementia (or other neurodegenerative condition). In another example, the logged time from ear to charger for a user may be used to track a new hearing device user's progress at using the device. For example, the new hearing device user may be notified of positive progress with using the hearing device. In addition or alternatively, a clinician may be notified that the new hearing device user is having trouble (not improving or getting worse) with device insertion or placement in a charger. In some examples, the recorded data or feedback from insertion may be used in programming the device by an audiologist. For example, if the audiologist knows that it takes a particular user more or less time than an average user to transfer the device from the charger to their ear, the audiologist may program the device to boot up slower or quicker for that user.

In one example, the time/duration and sensor data is logged in a hearing device. The time/duration and sensor data may be logged in external devices or in the cloud, in various examples. In some examples, when the hearing device is removed from the ear of the user, the system mutes the speaker of the device output (placing the device in an off state), but all other electronics in the device are still actively running such that motion data and timing can be recorded. In various examples, the present method and system may be programmed into firmware of the device. In other examples, a separate program or device may be used to track and record the sensor data and timing of device motion from ear to charger (or charger to ear) for the user.

In some examples, the present system may be used without a motion sensor (such as an IMU) and only use time/duration of the user's movement of the device from ear to charger (or charger to car). In one example, sensing the removal of a hearing device from one ear of the user, the hearing device in the opposite ear may play an audio prompt for the user to inform the user that measurement is in process, to focus the user on the task and to avoid distraction. In another example, the system may compare the user's duration and motions data between right and left devices. For example, tremor detection may be more robust when both devices of a user are tracked and analyzed. In various examples, other devices besides hearing devices may be used with the present system and method. For example, the present subject matter may be used on smart glasses, smart watches, and other electronic rechargeable wearable devices instead of ear worn devices to record user motion and timing from removal to charger placement. These other devices may have the present method programmed into firmware for auto on/off, recording of motion and/or timing data, and/or data analysis for tremor detection, in various examples. In another example, a smart phone may be used with the present system, and the motion data and/or timing data may be recorded from a user removing the device from a pocket and plugging in a charging cable or placing the smart phone on a wireless charger.

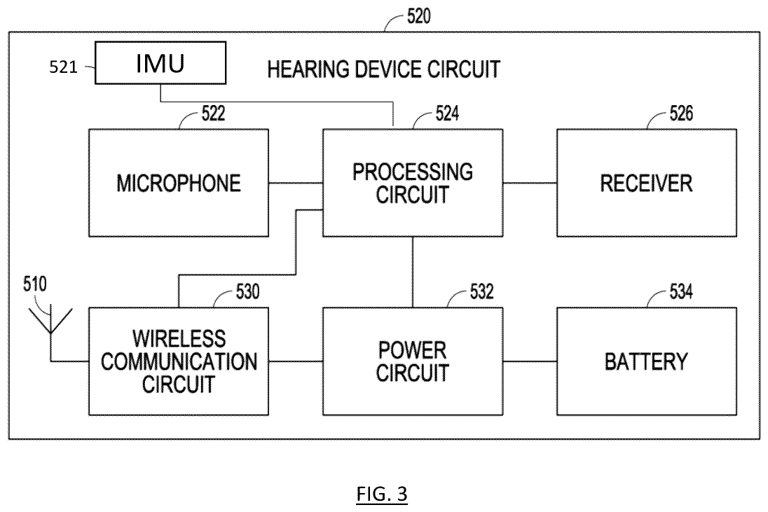

FIG. 3 illustrates a block diagram of a hearing device circuit, according to various examples of the present subject matter. Hearing device circuit 520 represents an example of portions of a hearing device and includes a microphone 522, a wireless communication circuit 530, an antenna 510, an inertial measurement unit (IMU) sensor 521, a processing circuit 524, a receiver (speaker) 526, a battery 534, and a power circuit 532. Microphone 522 receives sounds from the environment of the hearing device user (wearer of the hearing device). Wireless communication circuit 530 communicates with another device wirelessly using antenna 510, including receiving programming codes, streamed audio signals, and/or other audio signals and transmitting programming codes, audio signals, and/or other signals. Examples of the other device includes other hearing devices of other users, another hearing device of a pair of hearing devices for the same wearer, a hearing device host device, an assistive listening device (ALD), an audio streaming device, a smartphone, and other devices capable of communicating with hearing devices wirelessly. Processing circuit 524 controls the operation of a hearing device using the programming codes and processes the sounds received by microphone 522 and/or the audio signals received by wireless communication circuit 530 to produce output sounds. Receiver 526 transmits output sounds to an ear canal of the hearing device wearer. Battery 534 and power circuit 532 constitute the power source for the operation of hearing device circuit 520. In one example, power circuit 532 can include a power management circuit. In another alternative or additional example, battery 534 can include a rechargeable battery and power circuit 532 can include a recharging circuit for recharging the rechargeable battery.

In various examples, the hearing device is configured for tremor detection. The hearing device circuit 520 includes at least one processor or processing circuit 524 and data storage in communication with the processing circuit 524. The data storage comprises instructions thereon that, when executed by the processing circuit 524, causes the processing circuit 524 to perform the functions of the present systems and methods. For example, the processing circuit may perform operations to: receive a signal from the IMU sensor 521, begin recording the signal from the IMU sensor upon determining the hearing device has been removed from an ear of a user, discontinue recording the signal from the IMU sensor in the memory (or data storage) upon determining the hearing device has been placed on or in a charging device or on a surface, use the recorded signal to determine whether the user has a tremor, and provide a notification indicative of the presence of the tremor to one or more of the user or a clinician upon determining the user has the tremor. While the IMU sensor 521 is depicted within the hearing device, the IMU sensor 521 may be outside the device, incorporated into a housing of the device, or in any other position inside or outside the device. The hearing device circuit 520 may be included in an ear bud, headphones, a hearing aid, or other ear-wearable device, in various examples.

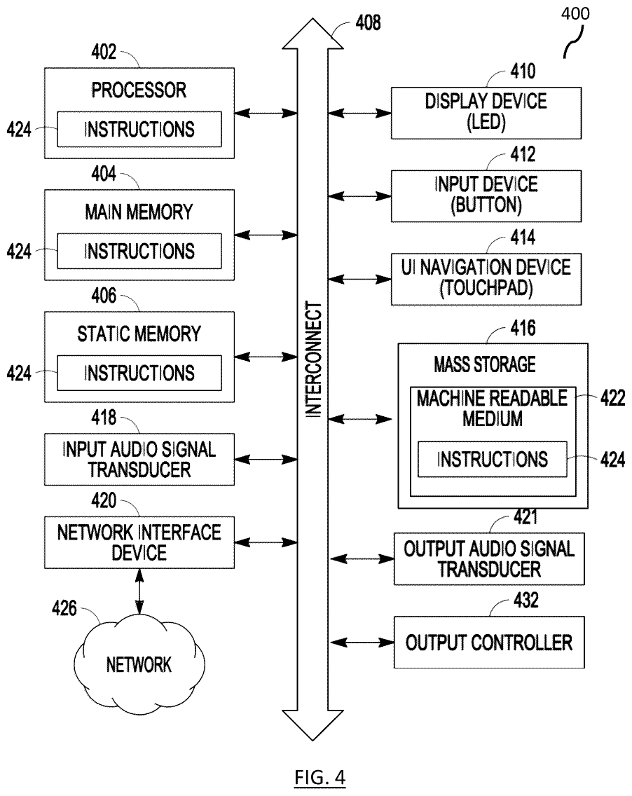

FIG. 4 illustrates a block diagram of an example machine 400 upon which any one or more of the techniques (e.g., methodologies) discussed herein may perform. In alternative examples, the machine 400 may operate as a standalone device or may be connected (e.g., networked) to other machines. In a networked deployment, the machine 400 may operate in the capacity of a server machine, a client machine, or both in server-client network environments. In an example, the machine 400 may act as a peer machine in peer-to-peer (P2P) (or other distributed) network environment. The machine 400 may be a personal computer (PC), a tablet PC, a set-top box (STB), a personal digital assistant (PDA), a mobile telephone, a hearing device, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specified operations when operating. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine (e.g., computer system) 400 may include a hardware processor 402 (e.g., a central processing unit (CPU), a graphics processing unit (GPU), a hardware processor core, or any combination thereof), a main memory 404, and a static memory 406, some or all of which may communicate with each other via an interlink (e.g., bus) 408. The machine 400 may further include a display unit 410, an alphanumeric input device 412 (e.g., a keyboard), and a user interface (UI) navigation device 414 (e.g., a mouse). In an example, the display unit 410, input device 412, and UI navigation device 414 may be a touch screen display. The machine 400 may additionally include a storage device (e.g., drive unit) 416, one or more input audio signal transducers 418 (e.g., microphone), a network interface device 420, and one or more output audio signal transducers 421 (e.g., speaker). The machine 400 may include an output controller 432, such as a serial (e.g., universal serial bus (USB), parallel, or other wired or wireless (e.g., infrared (IR), near-field communication, etc.) connection to communicate or control one or more peripheral devices (e.g., a printer, card reader, etc.).

The storage device 416 may include a machine readable medium 422 on which is stored one or more sets of data structures or instructions 424 (e.g., software) embodying or utilized by any one or more of the techniques or functions described herein. The instructions 424 may also reside, completely or at least partially, within the main memory 404, within static memory 406, or within the hardware processor 402 during execution thereof by the machine 400. In an example, one or any combination of the hardware processor 402, the main memory 404, the static memory 406, or the storage device 416 may constitute machine readable media.

While the machine readable medium 422 is illustrated as a single medium, the term “machine readable medium” may include a single medium or multiple media (e.g., a centralized or distributed database, and/or associated caches and servers) configured to store the one or more instructions 424.

The term “machine readable medium” may include any medium that is capable of storing, encoding, or carrying instructions for execution by the machine 400 and that cause the machine 400 to perform any one or more of the techniques of the present disclosure, or that is capable of storing, encoding or carrying data structures used by or associated with such instructions. Non-limiting machine-readable medium examples may include solid-state memories, and optical and magnetic media. In an example, a massed machine-readable medium comprises a machine-readable medium with a plurality of particles having invariant (e.g., rest) mass. Accordingly, massed machine-readable media are not transitory propagating signals. Specific examples of massed machine-readable media may include: non-volatile memory, such as semiconductor memory devices (e.g., Electrically Programmable Read-Only Memory (EPROM), Electrically Erasable Programmable Read-Only Memory (EEPROM) and flash memory devices; magnetic disks, such as internal hard disks and removable disks; magneto-optical disks; and CD-ROM and DVD-ROM disks.

The instructions 424 may further be transmitted or received over a communications network 426 using a transmission medium via the network interface device 420 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 420 may include one or more physical jacks (e.g., Ethernet, coaxial, or phone jacks) or one or more antennas to connect to the communications network 426. In an example, the network interface device 420 may include a plurality of antennas to communicate wirelessly using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 400, and includes digital or analog communications signals or other intangible medium to facilitate communication of such software.

Various examples of the present subject matter support wireless communications with a hearing device. In various examples the wireless communications may include standard or nonstandard communications. Some examples of standard wireless communications include link protocols including, but not limited to, Bluetooth™, BLE, Auracast, IEEE 802.11 (wireless LANs), 802.15 (WPANs), 802.16 (WiMAX), cellular protocols including, but not limited to CDMA and GSM, ZigBee, and ultra-wideband (UWB) technologies. Such protocols support radio frequency communications and some support infrared communications while others support NFMI. Although the present system is demonstrated as a radio system, it is possible that other forms of wireless communications may be used such as ultrasonic, optical, infrared, and others. It is understood that the standards which may be used include past and present standards. It is also contemplated that future versions of these standards and new future standards may be employed without departing from the scope of the present subject matter.

The wireless communications support a connection from other devices. Such connections include, but are not limited to, one or more mono or stereo connections or digital connections having link protocols including, but not limited to 802.3 (Ethernet), 802.4, 802.5, USB, SPI, PCM, ATM, Fibre-channel, Firewire or 1394, InfiniBand, or a native streaming interface. In various examples, such connections include all past and present link protocols. It is also contemplated that future versions of these protocols and new future standards may be employed without departing from the scope of the present subject matter.

Hearing assistance devices typically include at least one enclosure or housing, a microphone, hearing assistance device electronics including processing electronics, and a speaker or “receiver.” Hearing assistance devices may include a power source, such as a battery. In various examples, the battery is rechargeable. In various examples multiple energy sources are employed. It is understood that in various examples the microphone is optional. It is understood that in various examples the receiver is optional. It is understood that variations in communications protocols, antenna configurations, and combinations of components may be employed without departing from the scope of the present subject matter. Antenna configurations may vary and may be included within an enclosure for the electronics or be external to an enclosure for the electronics. Thus, the examples set forth herein are intended to be demonstrative and not a limiting or exhaustive depiction of variations.

It is understood that digital hearing assistance devices include a processor. In digital hearing assistance devices with a processor, programmable gains may be employed to adjust the hearing assistance device output to a wearer's particular hearing impairment. The processor may be a digital signal processor (DSP), microprocessor, microcontroller, other digital logic, or combinations thereof. The processing may be done by a single processor, or may be distributed over different devices. The processing of signals referenced in this application may be performed using the processor or over different devices. Processing may be done in the digital domain, the analog domain, or combinations thereof. Processing may be done using subband processing techniques. Processing may be done using frequency domain or time domain approaches. Some processing may involve both frequency and time domain aspects. For brevity, in some examples drawings may omit certain blocks that perform frequency synthesis, frequency analysis, analog-to-digital conversion, digital-to-analog conversion, amplification, buffering, and certain types of filtering and processing. In various examples of the present subject matter the processor is adapted to perform instructions stored in one or more memories, which may or may not be explicitly shown. Various types of memory may be used, including volatile and nonvolatile forms of memory. In various examples, the processor or other processing devices execute instructions to perform a number of signal processing tasks. Such examples may include analog components in communication with the processor to perform signal processing tasks, such as sound reception by a microphone, or playing of sound using a receiver (i.e., in applications where such transducers are used). In various examples of the present subject matter, different realizations of the block diagrams, circuits, and processes set forth herein may be created by one of skill in the art without departing from the scope of the present subject matter.

It is further understood that different hearing devices may embody the present subject matter without departing from the scope of the present disclosure. The devices depicted in the figures are intended to demonstrate the subject matter, but not necessarily in a limited, exhaustive, or exclusive sense. It is also understood that the present subject matter may be used with a device designed for use in the right ear or the left ear or both ears of the wearer.

The present subject matter is demonstrated for hearing devices, including hearing assistance devices, including but not limited to, behind-the-ear (BTE), in-the-ear (ITE), in-the-canal (ITC), receiver-in-canal (RIC), invisible-in-canal (IIC) or completely-in-the-canal (CIC) type hearing assistance devices. It is understood that behind-the-ear type hearing assistance devices may include devices that reside substantially behind the ear or over the ear. Such devices may include hearing assistance devices with receivers associated with the electronics portion of the behind-the-ear device, or hearing assistance devices of the type having receivers in the ear canal of the user, including but not limited to RIC or receiver-in-the-ear (RITE) designs. The present subject matter may also be used in hearing assistance devices generally, such as cochlear implant type hearing devices. The present subject matter may also be used in deep insertion devices having a transducer, such as a receiver or microphone. The present subject matter may be used in bone conduction or otherwise osseointegrated hearing devices, in some examples. The present subject matter may be used in devices whether such devices are standard or custom fit and whether they provide an open or an occlusive design. It is understood that other hearing devices not expressly stated herein may be used in conjunction with the present subject matter.

### Other Notes and Examples

Example 1 is a hearing device including a housing, a motion sensor on or in the housing, hearing electronics within the housing, the hearing electronics including at least one processor and a memory including instructions that, when executed by the at least one processor, cause the at least one processor to perform operations to: receive a signal from the motion sensor, upon determining the hearing device has been removed from an ear of a user, begin recording the signal from the motion sensor, upon determining the hearing device has been placed on or in a charging device or on a surface, discontinue recording the signal from the motion sensor in the memory, use the recorded signal to determine whether the user has a tremor, and upon determining the user has the tremor, provide a notification indicative of the presence of the tremor to one or more of the user or a clinician.

In Example 2, the subject matter of Example 1 optionally includes wherein determining when the hearing device has been removed from the ear includes detecting a change in acoustic environment of a microphone of the hearing device.

In Example 3, the subject matter of Example 2 optionally includes wherein the change in acoustic environment includes occlusion or covering up of the microphone.

In Example 4, the subject matter of Example 2 or Example 3 optionally includes wherein the change in acoustic environment includes feedback from the microphone.

In Example 5, the subject matter of any of Examples 1-4 optionally includes wherein determining when the hearing device has been removed from the ear includes using the signal from the motion sensor.

In Example 6, the subject matter of any of Examples 1-5 optionally includes wherein determining when the hearing device has been removed from the ear includes using a received signal from a proximity sensor.

In Example 7, the subject matter of Example 6 optionally includes wherein the proximity sensor includes an optical sensor.

In Example 8, the subject matter of any of Examples 1-7 optionally includes wherein determining when the hearing device has been placed on or in the charging device includes detecting when charging contacts of the hearing device touch contacts of the charging device.

In Example 9, the subject matter of any of Examples 1-8 optionally includes wherein using the recorded signal to determine whether the user has the tremor includes comparing the recorded signal to a baseline signal for the user.

In Example 10, the subject matter of any of Examples 1-9 optionally includes wherein the hearing device includes a hearing aid.

Example 11 is a method including determining when a hearing device has been removed from an ear of a user, receiving a signal from an motion sensor on or in a housing of the hearing device, upon determining the hearing device has been removed, beginning recording the signal from the motion sensor in a memory, determining when the hearing device has been placed on or in a charging device, upon determining the hearing device has been placed on or in the charging device, discontinuing recording the signal from the motion sensor in the memory, analyzing the recorded signal to determine whether the user has a tremor, and upon determining the user has the tremor, providing a notification indicative of the presence of the tremor to one or more of the user or a clinician.

In Example 12, the subject matter of Example 11 optionally includes wherein determining when the hearing device has been removed from the ear includes detecting a change in acoustic environment of a microphone of the hearing device.

In Example 13, the subject matter of Example 12 optionally includes wherein the change in acoustic environment includes occlusion or covering up of the microphone.

In Example 14, the subject matter of Example 12 or Example 13 optionally includes wherein the change in acoustic environment includes feedback from the microphone.

In Example 15, the subject matter of any of Examples 11-14 optionally includes wherein determining when the hearing device has been removed from the ear includes using the signal from the motion sensor.

In Example 16, the subject matter of any of Examples 11-15 optionally includes wherein determining when the hearing device has been removed from the ear includes using a received signal from a proximity sensor.

In Example 17, the subject matter of Example 16 optionally includes wherein the proximity sensor includes an optical sensor.

In Example 18, the subject matter of any of Examples 11-17 optionally includes wherein determining when the hearing device has been placed on or in the charging device includes detecting when charging contacts of the hearing device touch contacts of the charging device.

In Example 19, the subject matter of any of Examples 11-18 optionally further includes starting a timer upon determining the hearing device has been removed, ending the timer upon determining the hearing device has been placed on or in the charging device, comparing the timer to a predetermined time limit, and upon determining that the timer exceeds the predetermined time limit, discarding the stored signal from the motion sensor.

In Example 20, the subject matter of any of Examples 11-19 optionally includes wherein the hearing device includes a hearing aid.

Example 21 is at least one machine-readable medium including instructions that, when executed by processing circuitry, cause the processing circuitry to perform operations to implement of any of Examples 1-20.

Example 22 is an apparatus comprising means to implement of any of Examples 1-20.

Example 23 is a system to implement of any of Examples 1-20.

Example 24 is a method to implement of any of Examples 1-20.

This application is intended to cover adaptations or variations of the present subject matter. It is to be understood that the above description is intended to be illustrative, and not restrictive. The scope of the present subject matter should be determined with reference to the appended claims, along with the full scope of legal equivalents to which such claims are entitled.

What is claimed is:

1. A hearing device comprising:
a housing;
an inertial measurement unit (IMU) sensor on or in the housing;
hearing electronics within the housing, the hearing electronics including at least one processor and a memory including instructions that, when executed by the at least one processor, cause the at least one processor to perform operations to:
receive a signal from the IMU sensor;
upon determining the hearing device has been removed from an ear of a user, begin recording the signal from the IMU sensor;
upon determining the hearing device has been placed on or in a charging device or on a surface, discontinue recording the signal from the IMU sensor in the memory;
use the recorded signal to determine whether the user has a tremor; and
upon determining the user has the tremor, provide a notification indicative of the presence of the tremor to one or more of the user or a clinician.

2. The hearing device of claim 1, wherein determining when the hearing device has been removed from the ear includes detecting a change in acoustic environment of a microphone of the hearing device.

3. The hearing device of claim 2, wherein the change in acoustic environment includes occlusion or covering up of the microphone.

4. The hearing device of claim 2, wherein the change in acoustic environment includes feedback from the microphone.

5. The hearing device of claim 1, wherein determining when the hearing device has been removed from the ear includes using the signal from the IMU sensor.

6. The hearing device of claim 1, wherein determining when the hearing device has been removed from the ear includes using a received signal from a proximity sensor.

7. The hearing device of claim 6, wherein the proximity sensor includes an optical sensor.

8. The hearing device of claim 1, wherein determining when the hearing device has been placed on or in the charging device includes detecting when charging contacts of the hearing device touch contacts of the charging device.

9. The hearing device of claim 1, wherein using the recorded signal to determine whether the user has the tremor includes comparing the recorded signal to a baseline signal for the user.

10. The hearing device of claim 1, wherein the hearing device includes a hearing aid.

11. A method comprising:
determining when a hearing device has been removed from an ear of a user;
receiving a signal from an inertial measurement unit (IMU) sensor on or in a housing of the hearing device;
upon determining the hearing device has been removed, beginning recording the signal from the IMU sensor in a memory;
determining when the hearing device has been placed on or in a charging device;
upon determining the hearing device has been placed on or in the charging device, discontinuing recording the signal from the IMU sensor in the memory;
analyzing the recorded signal to determine whether the user has a tremor; and
upon determining the user has the tremor, providing a notification indicative of the presence of the tremor to one or more of the user or a clinician.

12. The method of claim 11, wherein determining when the hearing device has been removed from the ear includes detecting a change in acoustic environment of a microphone of the hearing device.

13. The method of claim 12, wherein the change in acoustic environment includes occlusion or covering up of the microphone.

14. The method of claim 12, wherein the change in acoustic environment includes feedback from the microphone.

15. The method of claim 11, wherein determining when the hearing device has been removed from the ear includes using the signal from the IMU sensor.

16. The method of claim 11, wherein determining when the hearing device has been removed from the ear includes using a received signal from a proximity sensor.

17. The method of claim 16, wherein the proximity sensor includes an optical sensor.

18. The method of claim 11, wherein determining when the hearing device has been placed on or in the charging device includes detecting when charging contacts of the hearing device touch contacts of the charging device.

19. The method of claim 11, further comprising:
starting a timer upon determining the hearing device has been removed;
ending the timer upon determining the hearing device has been placed on or in the charging device;
comparing the timer to a predetermined time limit; and
upon determining that the timer exceeds the predetermined time limit, discarding the stored signal from the IMU sensor.

20. The method of claim 11, wherein the hearing device includes a hearing aid.
